(19) United States
(12) Patent Application Publication
(10) Pub. No.: US 2022/0230736 A1
(43) Pub. Date: Jul. 21, 2022

(54) SYSTEM AND METHOD FOR ON-DEMAND ONLINE CONSULTATION

(71) Applicant: Teladentist PC Inc., North Las Vegas, NV (US)

(72) Inventor: Vilas Sastry, Las Vegas, NV (US)

(21) Appl. No.: 17/154,981

(22) Filed: Jan. 21, 2021

(51) Int. Cl. G16H 40/20; G06Q 10/10; G16H 80/00

(52) U.S. Cl. CPC G16H 40/20; G16H 80/00; G06Q 10/1095

(57) ABSTRACT

A healthcare network system to provide on-demand online consultation is provided. The healthcare network system is configured to monitor an appointment schedule of a plurality of healthcare providers by receiving real-time appointment data and receive a plurality of consultation requests from a corresponding plurality of users. Further, the system is configured to identify a set of available healthcare providers in response to the consultation request; wherein the available healthcare provider is selected based on a set of user parameters and transmit a consultation request to a set of available healthcare providers and receive an acceptance response from a subset of healthcare providers. Further, the subset of healthcare providers is provided to the user and enable the user to select one healthcare provider. The system then facilitates a consultation between the healthcare provider and the user in a virtual consultation room.

### BACKGROUND

The invention generally relates to healthcare platforms and more particularly to a system and method for on-demand online consultation with a healthcare provider.

For most people, visiting the doctor is a long and arduous endeavor, often requiring an individual to schedule his or her appointment weeks or months in advance. Even when an individual is mildly ill, he or she may feel that it is unnecessary to visit the doctor due to the unbelievably long wait times and the need to physically be present at the doctor's office. For instance, the individual may merely want to ask a simple medical question such as which over-the-counter medication is best for his or her ailment. As a result, the patient may feel it is unnecessary to take time out of his or her busy schedule or take time off from work to receive such simple medical advice.

Virtual healthcare systems have addressed the issue to some extent by cutting down the travel time of the individual to almost nothing. Further, since the consultation can be done from the comfort of one's home, the wait time for the healthcare provider is not as long as it would have been at the doctor's office.

In most online healthcare consultation practices, getting an appointment with a preferred healthcare provider is a challenging task. Sometimes, the individual has to settle for the earliest available slot of the healthcare provider he or she seeks. Another way to get an appointment is to call a front desk personnel of the healthcare provider, who then schedules a virtual visit with a provider based on probable availability.

The above described common practices to get a healthcare appointment with a preferred healthcare provider is tedious and time consuming. Also, in the event of an emergency, patients oftentimes head to an urgent care center and are reluctant to consult using an online virtual consult platform although it is much easier and convenient, and may have been able to solve their issue faster. Practices who schedule such consultations often times experience a high no show rate due to the fact that the patient may have sought care elsewhere in the meantime.

Therefore, what is needed is, a healthcare system that is able to connect an individual to a set of available healthcare providers and enable the individual to select the healthcare provider based on need and availability.

### SUMMARY

Briefly according to one embodiment of the present technique, a healthcare network system for managing on-demand consultations with a healthcare provider is disclosed. The healthcare network system to monitor an appointment schedule of a plurality of healthcare providers by receiving real-time appointment data and receive a plurality of consultation requests from a corresponding plurality of users. Further, the system is configured to identify a set of available healthcare providers in response to the consultation request; wherein the available healthcare provider is selected based on a set of user parameters and transmit a consultation request to a set of available healthcare providers and receive an acceptance response from a subset of healthcare providers. Further, the subset of healthcare providers is provided to the user and enable the user to select one healthcare provider. The system then facilitates a consultation between the healthcare provider and the user in a virtual consultation room.

In another embodiment, a method for providing on-demand online healthcare consultation service is provided. The method comprises providing, a user interface comprising a multistate selection feature that enables a user to raise a consultation request, mapping the consultation request to a corresponding set of healthcare providers that are available for consultation and determining a subset of available healthcare providers available to attend the consultation request. The method further includes providing the subset of available healthcare providers to the user via the user interface, enabling the user to select a healthcare provider; and transmitting a scheduling request to the selected healthcare provider.

### BRIEF DESCRIPTION OF THE FIGURES

These and other features, aspects, and advantages of the example embodiments will become better understood when the following detailed description is read with reference to the accompanying drawings in which like characters represent like parts throughout the drawings, wherein:

FIG. 1 is a block diagram of one embodiment of a healthcare network system implemented according to an aspect of the present technique;

FIG. 2 is a flow chart of one method by which an on-demand consultation is implemented according to aspects of the present technique;

FIG. 3 is a flow chart for one method by which a user requests a consultation with a healthcare network system, implemented according to aspects of the present technique;

FIG. 4 and FIG. 5 is a graphical representation of the manner in which consultation requests are transmitted and accept messages are received by the user according to aspects of the present technique;

FIG. 6 through FIG. 9 are example user interfaces provided to a user via, implemented according to various aspects of the present technique; and

FIG. 10 is a block diagram illustrating an example computer system, according to some aspects of the present description.

### DETAILED DESCRIPTION OF EXAMPLE EMBODIMENTS

Various example embodiments will now be described more fully with reference to the accompanying drawings in which only some example embodiments are shown. Specific structural and functional details disclosed herein are merely representative for purposes of describing example embodiments. Example embodiments, however, may be embodied in many alternate forms and should not be construed as limited to only the example embodiments set forth herein. On the contrary, example embodiments are to cover all modifications, equivalents, and alternatives thereof.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed but may also have additional steps not included in the figures. It should also be noted that in some alternative implementations, the functions/acts/steps noted may occur out of the order noted in the figures. For example, two figures shown in succession may, in fact, be executed substantially concurrently or may sometimes be executed in the reverse order, depending upon the functionality/acts involved.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or a section. Thus, a first element, component, region, layer, or section discussed below could be termed a second element, component, region, layer, or section without departing from the scope of example embodiments.

Spatial and functional relationships between elements (for example, between modules) are described using various terms, including “connected,” “engaged,” “interfaced,” and “coupled.” Unless explicitly described as being “direct,” when a relationship between first and second elements is described in the description below, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular example embodiments only and is not intended to be limiting. Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Unless specifically stated otherwise, or as is apparent from the description, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

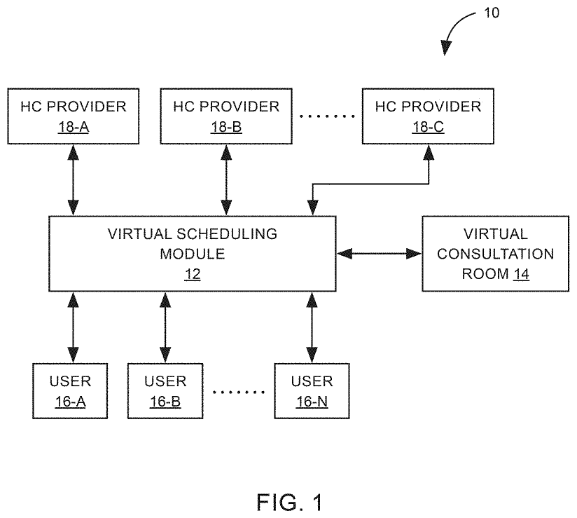

Example embodiments of the present description provide a system and method for on-demand online consultation with a healthcare provider. FIG. 1 is a block diagram of one embodiment of a healthcare network system implemented according to an aspect of the present technique. The healthcare network system 10 comprises a virtual scheduling module 12 and a virtual consultation room 14. The healthcare network systems is coupled to several users as depicted by 16-a, 16-b, 16-c through 16-n. Similarly, the healthcare network system is also coupled to a plurality of healthcare providers as depicted by 18-a, 18-b, 18-c through 18-n. Each block is described in further detail below.

Virtual scheduling module 12 is configured to monitor an appointment schedule of a plurality of healthcare providers by receiving real-time appointment data. In one embodiment, the appointment data is updated daily. As used herein appointment data refers to calendar schedules of multiple healthcare providers coupled to the healthcare network system. Examples of healthcare providers include medical practitioners such as doctors, dentists, physiotherapists, psychologists, sports medicine practitioners, and the like.

The virtual scheduling module 12 is configured to receive a plurality of consultation requests from a corresponding plurality of users. In one embodiment, the users may connect with the healthcare network system by installing an application on a computing device such as a mobile phone. The application enables the user to transmit a consultation request to the virtual scheduling module.

Virtual scheduling module 12 is configured to extract a set of parameters from the consultation. In one embodiment, the consultation request includes information related to the user such as the user profile data which includes typically the user's name, age, gender, etc. Further, the consultation request comprises a preferred time slot indicating when the user would like to consult with a healthcare provider. The consultation request further includes the user's location data. Further the consultation request may include at least one symptom data related to a symptom that is being experienced by the user.

The virtual scheduling module 12 is further configured identify a set of available healthcare providers based on the set of parameters extracted from the consultation request. In one embodiment, the available healthcare providers is selected based the location of the user. In a further embodiment, the virtual scheduling monitor selects healthcare providers that are available at the earliest time slot indicated by the user.

The virtual scheduling module 12 is configured to transmit an appointment request to a set of available healthcare providers. In one embodiment, the set of available healthcare providers is identified by extracting availability data from the real-time appointment data and matching it with the set of parameters extracted from the consultation request. In one embodiment the appointment request specifies the user's profile data, the user's location and one or more symptoms that the user is experiencing. The information helps the healthcare provider to determine if they can attend to the user's consultation request.

The healthcare providers that accept the appointment request then transmits an accept response to the virtual scheduling module 12. Upon receiving an accept response from a subset of available healthcare providers, the virtual scheduling module relays the subset of available healthcare providers to the user. The user then selects one healthcare provider to proceed with his/her consultation request. The virtual scheduling module then blocks the time of the selected healthcare provider for a virtual consultation with the user. Further, the virtual scheduling module also notifies the remaining set of healthcare providers that their services have been declined thereby freeing up their time for the next consultation request. Thus, the appointment schedule of a plurality of healthcare providers registered with the healthcare network system is updated real-time.

Virtual consultation room 14 is configured to facilitate a consultation between the healthcare provider and the user in a virtual consultation room. In one embodiment, the virtual consultation room 14 enables the user to interact with the healthcare provider by a plurality of interactive tools. Examples of interactive tools include a microphone, text interface, video conferencing, and the like. The manner in which the healthcare

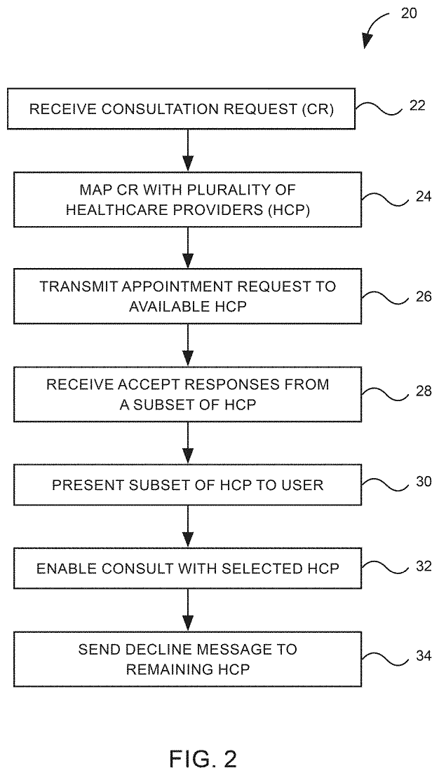

FIG. 2 is a flow chart of one method by which an on-demand consultation is implemented according to aspects of the present technique. The process is explained below with reference to one user accessing a healthcare network system as described in FIG.1. IT may be noted that the healthcare network system is capable of handling multiple users simultaneously and is configured to schedule multiple consultations for its users.

The process starts when the user requests a consultation with any healthcare provider listed on the healthcare network system as described above. An example of a healthcare provider is a clinic that includes several healthcare specialists such as pediatricians, gastroenterologists, general practitioners, dentists, and the like. It may be noted that user may access the healthcare network system using any computing device such a computer system, a handheld device such as a smartphone or a tablet, etc. The manner in which the healthcare network systems enables the user to connect with a healthcare provider is listed below.

At step 22, a consultation request is received by the healthcare network system. In one embodiment, the consultation request comprises a preferred time slot indicating when the user would like to consult with a healthcare provider. In one embodiment, the consultation request may include health related information such as at least one symptom data related to a symptom that is being experienced by the user. In another embodiment, the consultation request also specifies the location at which the user is at the present moment.

At step 24, the consultation request is mapped with a plurality of available healthcare providers registered with the healthcare network system. As used herein, an available healthcare provider refers to a healthcare provider who is available for an online consultation at the time specified in the consultation request. In one embodiment, the available healthcare providers within a specific distance, for example, 3 miles, is matched. The availability of the healthcare providers is dynamically updated by the healthcare network system in real time.

At step 26, an appointment request is transmitted to the plurality of available healthcare providers. In one embodiment, the appointment request is transmitted as a notification the healthcare providers on their handheld device.

At step 28, a plurality of accept responses is received from a subset of available healthcare providers accepting to the appointment request. At step 30, the subset of available healthcare providers is presented to the user to enable said user to select the healthcare providers he/she wishes to consult. In one embodiment, the available healthcare providers is presented in a list format with the nearest healthcare provider listed on the top. In another embodiment, the available healthcare providers are presented on a map to enable the user to visualize a location of each available healthcare provider.

At step 32, the user selects one healthcare provider from the list of healthcare providers. The user is then guided into a virtual consultation room where he/she can connect with the healthcare provider for a virtual consult. At step 34, a decline message is sent to the remaining healthcare providers to inform them that the consultation request is closed and thereby freeing up their appointment schedule for the another consultation request.

The above described figure is with reference to how the healthcare network system routes consultation requests received from multiple users. The manner in which the user raises a consultation request is described in detail below.

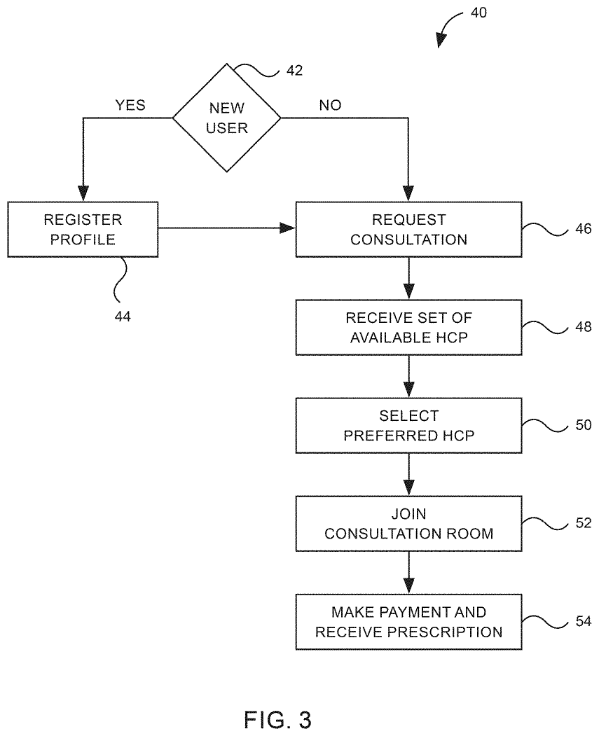

FIG. 3 is a flow chart for one method by which a user requests a consultation with a healthcare network system, implemented according to aspects of the present technique. The process starts when a user raises a consultation request with the the healthcare network system as described with reference to FIG. 1. The manner in which the healthcare network systems enables the user to connect with a healthcare provider is listed below.

At step 42, the user accesses the healthcare network system via a user interface. The user interface provides a multistate selection feature that enables a user to raise a consultation request. In one embodiment, the user can access the healthcare network system by logging on to the registered website or by installing an application on a handheld device. If the user is a new user, the healthcare network system will request the user to register his/her profile as show in step 44.

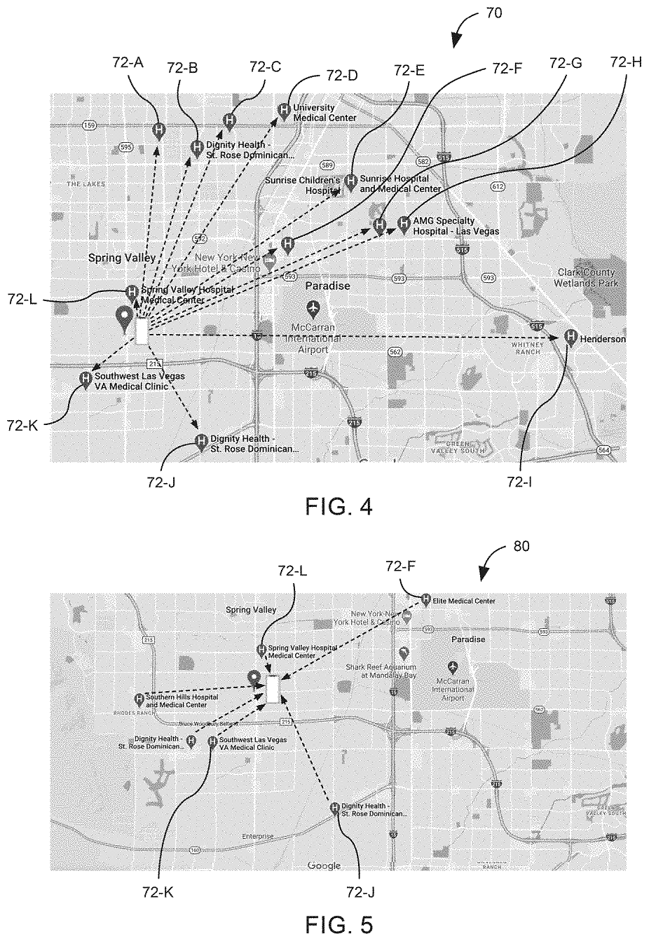

At step 46, the user requests for a consultation with an available healthcare provider listed in the healthcare network system. In one embodiment, the user provides information regarding one or more symptoms that is being experienced at the time. The healthcare network system transmits an appointment request to a set of available healthcare providers as shown in FIG. 4. As can be seen in FIG. 4, the appointment request is sent to a plurality of healthcare providers indicated by reference numerals 72-A through 72L. In this embodiment, the healthcare providers are within a close proximity to the user's location indicated by the reference numeral 74.

At step 48, the user receives an accept response from a subset of available healthcare provider listed in the healthcare network system as shown in FIG. 5 For example, the accept response is received from healthcare providers indicated by reference numerals 72-F, 72-J, 72-K and 72L. At step 50, the user selects any one of the healthcare providers from the subset of available healthcare provider.

At step 52, the user and the selected healthcare provider is guided into a virtual consultation room for a private consultation. The virtual consultation room has several interactive tools such a video conferencing, text messaging, etc. Before the end of consultation, the user is requested to make a payment for the completed consultation, subsequently receives a prescription and follow up instructions if any.

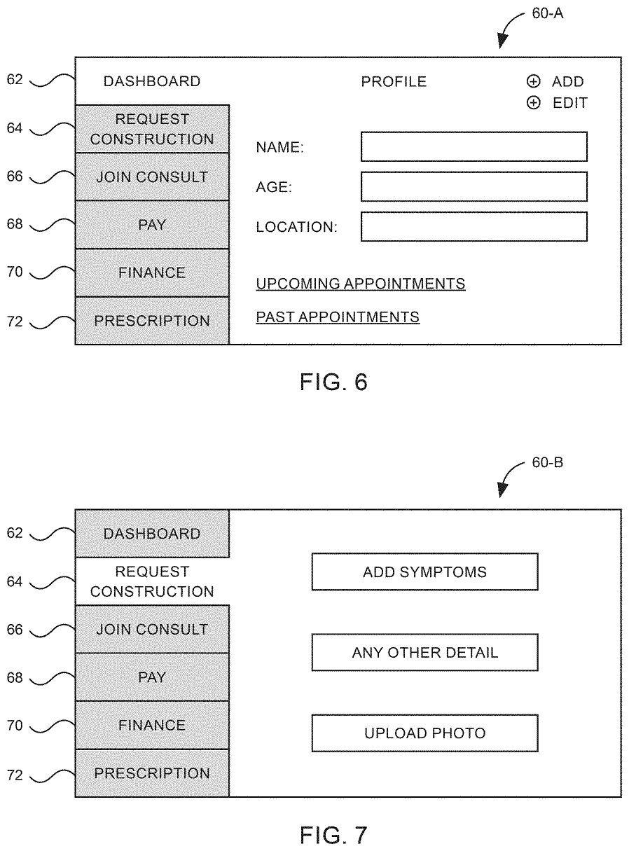

As noted in the above description, all steps in the process described in FIG. 3 are performed by a user via a user interface. The user interface allows the user to interactively with the healthcare network system easily right from raising a consultation request to interacting with a healthcare provider and also making a payment. Example user interfaces are described with reference to FIG. 6 through FIG. 9.

FIG. 6 is an example user interface that enables the user to provide specific details with reference to user's profile, according to aspects of the present technique. As can be seen in FIG. 6, user interface 60-A comprises tabs 62, 64, 66, 68, 70 and 72. When the user clicks on tab 62, a dashboard is presented where the user can add personal details such as the user's name, age, current location, etc. The user is also provided with links that provide details with reference to upcoming consultations and past consultations.

FIG. 7 is an example user interface that enables the user to request consultation, according to aspects of the present technique. As can be seen in FIG. 7, user interface 60-B comprises tabs 62, 64, 66, 68, 70 and 72. When the user clicks on tab 64, details of the consultation is presented to the user. The user may add one or more symptoms, provide any other details such as the duration of the symptoms or any other information that they would like to share with the healthcare provider. In addition, the user may also provide a photograph to provide visual details of the symptom, when required.

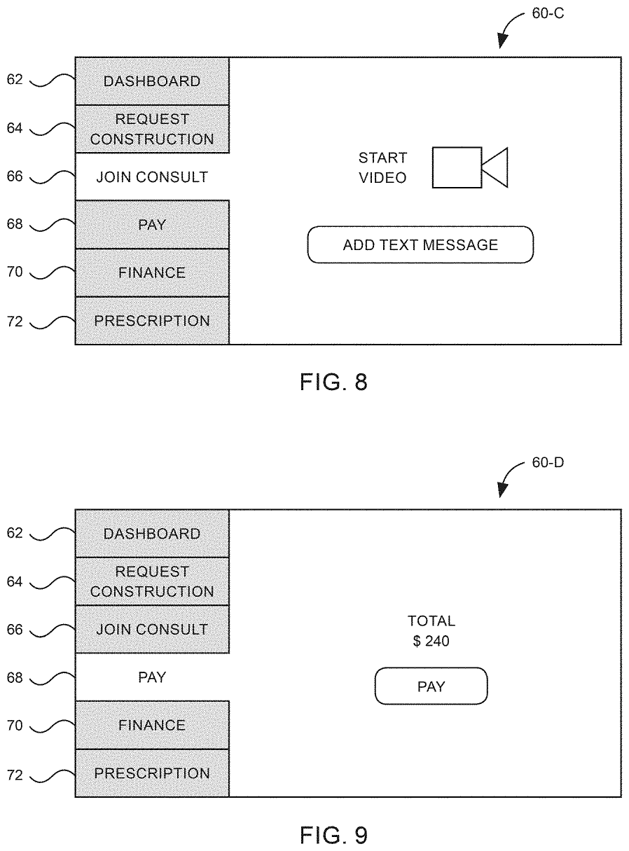

FIG. 8 is an example user interface that enables the user to join a consultation, according to aspects of the present technique. As can be seen in FIG. 8, user interface 60-C comprises tabs 62, 64, 66, 68, 70 and 72. When the user clicks on tab 66, a video link for a virtual consultation room is presented to the user. The user may also chat with the healthcare provider using text messaging.

FIG. 9 is an example user interface that enables the user to pay a consultation, according to aspects of the present technique. As can be seen in FIG. 8, user interface 60-C comprises tabs 62, 64, 66, 68, 70 and 72. When the user clicks on tab 68, a payment link for making a payment for the consultation is provided. The payment link may be further connected to the user's bank or electronic wallet using which the user may digitally pay for the consultation service. In addition, the user interface also provides a tab 70 for financing options available. Further, tab 72 provides the user with a prescription that the healthcare provider generates after the consultation.

The above-described invention provides several advantages to the user including providing a healthcare platform that connects the user to a set of available healthcare providers within a very short period of time thus avoiding the whole process of waiting for long periods before getting an appointment. Also, since the consultation is done online, the waiting period at the doctor's office is also avoided.

The systems and methods described herein may be partially or fully implemented by a special purpose computer system created by configuring a general-purpose computer to execute one or more particular functions embodied in computer programs. The functional blocks and flowchart elements described above serve as software specifications, which may be translated into the computer programs by the routine work of a skilled technician or programmer.

The computer programs include processor-executable instructions that are stored on at least one non-transitory computer-readable medium, such that when run on a computing device, cause the computing device to perform any one of the aforementioned methods. The medium also includes, alone or in combination with the program instructions, data files, data structures, and the like. Non-limiting examples of the non-transitory computer-readable medium include, but are not limited to, rewriteable non-volatile memory devices (including, for example, flash memory devices, erasable programmable read-only memory devices, or a mask read-only memory devices), volatile memory devices (including, for example, static random access memory devices or a dynamic random access memory devices), magnetic storage media (including, for example, an analog or digital magnetic tape or a hard disk drive), and optical storage media (including, for example, a CD, a DVD, or a Blu-ray Disc). Examples of the media with a built-in rewriteable non-volatile memory, include but are not limited to memory cards, and media with a built-in ROM, including but not limited to ROM cassettes, etc. Program instructions include both machine codes, such as produced by a compiler, and higher-level codes that may be executed by the computer using an interpreter. The described hardware devices may be configured to execute one or more software modules to perform the operations of the above-described example embodiments of the description, or vice versa.

Non-limiting examples of computing devices include a processor, a controller, an arithmetic logic unit (ALU), a digital signal processor, a microcomputer, a field programmable array (FPA), a programmable logic unit (PLU), a microprocessor, or any device which may execute instructions and respond. A central processing unit may implement an operating system (OS) or one or more software applications running on the OS. Further, the processing unit may access, store, manipulate, process, and generate data in response to the execution of software. It will be understood by those skilled in the art that although a single processing unit may be illustrated for convenience of understanding, the processing unit may include a plurality of processing elements and/or a plurality of types of processing elements. For example, the central processing unit may include a plurality of processors or one processor and one controller. Also, the processing unit may have a different processing configuration, such as a parallel processor.

The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

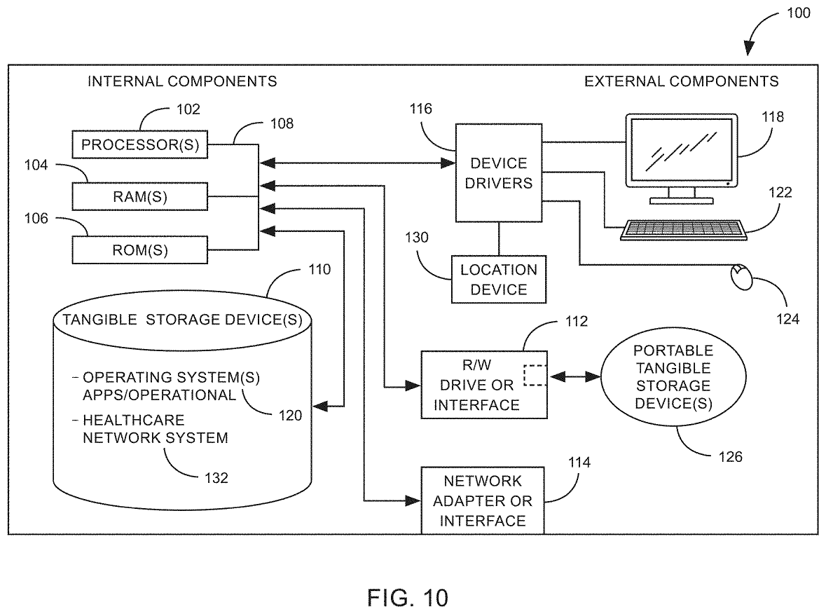

One example of a computing system 100 is described below in FIG. 10. The computing system 600 includes one or more processor 102, one or more computer-readable RAMs 104, and one or more computer-readable ROMs 106 on one or more buses 108. Further, the computer system 108 includes a tangible storage device 110 that may be used to execute operating systems 120 and the healthcare network system 132, an example of which is described in FIG. 1. Both, the operating system 120 and the healthcare network system 132 are executed by processor 102 via one or more respective RAMs 104 (which typically includes cache memory). The execution of the operating system 120 and/or the healthcare network system 132 by the processor 102, configures the processor 102 as a special-purpose processor configured to carry out the functionalities of the operation system 120 and/or the healthcare network system, as described above.

Examples of storage devices 110 include semiconductor storage devices such as ROM 506, EPROM, flash memory or any other computer-readable tangible storage device that may store a computer program and digital information.

Computing system 100 also includes a R/W drive or interface 112 to read from and write to one or more portable computer-readable tangible storage devices 126 such as a CD-ROM, DVD, memory stick or semiconductor storage device. Further, network adapters or interfaces 114 such as a TCP/IP adapter cards, wireless Wi-Fi interface cards, or 3G or 4G wireless interface cards or other wired or wireless communication links are also included in the computing system 100.

In one example embodiment, the healthcare network system 132 may be stored in tangible storage device 110 and may be downloaded from an external computer via a network (for example, the Internet, a local area network or another wide area network) and network adapter or interface 114.

Computing system 100 further includes device drivers 116 to interface with input and output devices. The input and output devices may include a computer display monitor 118, a keyboard 122, a keypad, a touch screen, a computer mouse 124, and/or some other suitable input device.

In this description, including the definitions mentioned earlier, the term ‘module’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware. The term code, as used above, may include software, firmware, and/or microcode, and may refer to programs, routines, functions, classes, data structures, and/or objects.

Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above. Shared memory hardware encompasses a single memory device that stores some or all code from multiple modules. Group memory hardware encompasses a memory device that, in combination with other memory devices, stores some or all code from one or more modules.

In some embodiments, the module may include one or more interface circuits. In some examples, the interface circuits may include wired or wireless interfaces that are connected to a local area network (LAN), the Internet, a wide area network (WAN), or combinations thereof The functionality of any given module of the present description may be distributed among multiple modules that are connected via interface circuits. For example, multiple modules may allow load balancing. In a further example, a server (also known as remote, or cloud) module may accomplish some functionality on behalf of a client module.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

While only certain features of several embodiments have been illustrated, and described herein, many modifications and changes will occur to those skilled in the art. It is, therefore, to be understood that the appended claims are intended to cover all such modifications and changes as fall within the true spirit of inventive concepts.

The afore mentioned description is merely illustrative in nature and is in no way intended to limit the disclosure, its application, or uses. The broad teachings of the disclosure may be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the example embodiments is described above as having certain features, any one or more of those features described with respect to any example embodiment of the disclosure may be implemented in and/or combined with features of any of the other embodiments, even if that combination is not explicitly described. In other words, the described example embodiments are not mutually exclusive, and permutations of one or more example embodiments with one another remain within the scope of this disclosure.

1. A healthcare network system for managing on-demand consultations with a healthcare provider, the system comprising:
a virtual scheduling module configured to:
monitor an appointment schedule of a plurality of healthcare providers by receiving real-time appointment data,
receive a plurality of consultation requests from a corresponding plurality of users;
transmit an appointment request to a plurality of healthcare providers based on the consultation and receive an accept response from a subset of available healthcare providers;
provide the subset of available healthcare providers to the user and enable the user to select one healthcare provider; and
a virtual consultation room configured to facilitate a consultation between the selected healthcare provider and the user in a virtual consultation room.

2. The healthcare network system of claim 1, wherein the virtual scheduling module is configured to transmit the appointment request by identifying a set of healthcare providers by extracting availability data from the real-time appointment data.

3. The healthcare network system of claim 2, wherein the virtual scheduling module is further configured to identify the set of available healthcare providers by extracting a set of parameters from the consultation request.

4. The healthcare network system of claim 3, wherein the consultation request comprises user profile data, a preferred consultation time slot, user location data and at least one symptom data related to a symptom that is being experienced by the user.

5. The healthcare network system of claim 1, wherein the virtual scheduling module is further configured to transmit a decline message to the subset of available healthcare providers except the selected healthcare provider.

6. The healthcare system of claim 1, wherein the virtual scheduling module is further configured to update the real-time appointment data and mark the selected healthcare provided as unavailable for a predefined period of time.

7. The healthcare system of claim 1, wherein the virtual consultation room enables the user to interact with the healthcare provider by a plurality of interactive tools; wherein the interactive tools support audio, video, text and combinations thereof.

8. A method for providing on-demand online healthcare consultation service, the method being performed by one or more processors and comprising:
providing, a user interface comprising a multistate selection feature that enables a user to raise a consultation request,
mapping the consultation request to a corresponding set of healthcare providers that are available for consultation,
determining a subset of available healthcare providers available to attend the consultation request;
providing the subset of available healthcare providers to the user via the user interface,
enabling the user to select a healthcare provider; and
transmitting a scheduling request to the selected healthcare provider.

9. The method of claim 8, further comprising providing a virtual consultation room via the user interface configured to enable the user to interact with the selected healthcare provider.

10. The method of claim 9, further comprising providing in the virtual consultation room, a plurality of interactive tools configured to enable the user to interact with the selected healthcare provider.

11. The method of claim 8, further comprising transmitting a decline message to the set of healthcare providers except the selected healthcare provider.

12. The method of claim 8, wherein the consultation request is mapped to healthcare providers with a pre-determined distance of a location of the user.

13. The method of claim 8, wherein the user is presented with a map representation of each location corresponding to the subset of healthcare providers.

14. The method of claim 8, wherein the user is presented with a list representation the subset of healthcare providers; wherein a nearest available healthcare provider is presented on the top of the list.

15. The method of claim 8, further comprising providing a payment option to the user to pay for the online consultation service.
